(19) United States
(12) Patent Application Publication
(10) Pub. No.: US 2004/0126797 A1
(43) Pub. Date: Jul. 1, 2004

(54) Micro-array system for micro amount reaction

(75) Inventors: Chih-Ying Hsu, Hsinchu (TW); Tzong-Hsiung Hseu, Hsinchu (TW); Ruey-Shing Huang, Hsinchu (TW); David R. Cox, Mountain View, CA (US); Chien-ming Wu, Hsinchu (TW); Jiann Heng Chen, Hsinchu (TW); Min-Lung Tsai, Hsinchu (TW); Ming-Tsong Lai, Hsinchu (TW); Yong-Li Pan, Hsinchu (TW); Tai-ching Fan, Hsinchu (TW)

Correspondence Address: William R. Evans, Ladas & Parry, 26 West 61 Street, New York, NY, 10023, US

(73) Assignees: NATIONAL TSING-HUA UNIVERSITY; STANFORD UNIVERSITY

(21) Appl. No.: 10/690,971

(22) Filed: Oct. 21, 2003

(30) Foreign Application Priority Data
Oct. 25, 2002 (TW) 091125053

(51) Int. Cl. C12Q 1/68; C12M 1/34

(52) U.S. Cl. 435/6; 435/287.2; 436/523

(57) ABSTRACT

The present invention relates to a micro-array system and the uses thereof. Particularly, the micro-array system is for a micro amount of biomolecules carrying on a bioreaction in a reaction solution, which comprises a substrate comprising a plurality of micro-wells for receiving the reaction solution; a plurality of micro-beads placing in the reaction solution for the biomolecules attached on surfaces thereon; and a vibrating module for vibrating the substrate, which makes the biomolecules attached on the micro-beads react evenly. Optionally, the micro-array system further comprises a temperature control module for controlling the temperature of the reaction solution. The bioreaction performed in the micro-array system according to the invention has the advantages of having a high sensitivity, being easily post-manipulated, being easily operated, having a high reliability and being reusable.

### BACKGROUND OF THE INVENTION

1. Field of the Invention

The present invention relates to a micro-array system.

2. Description of the Related Art

“Biochip” refers to a product to be applied in biochemical analysis on a substrate using micro-electro mechanical technology. The biochip provides many advantages, such as fast testing speed, needing small amounts of samples and agents, obtaining batch experiment data in one test, and is applicable in studying genes, proteins, cells, and tissues.

“DNA/protein micro-array chip” is an array of a set of DNAs/proteins with different sequences immobilized on a solid substrate as probes. Such chips are usually used in genetic mapping studies, mutational analysis, genome-wide monitoring of gene expression, drug discovery, disease diagnosis, etc. However, conventional micro-array chips have the problems of: (1) low sensitivity, especially when applied in the study of human biopsy (since the probes immobilized on a plate cannot react completely or evenly with the samples); (2) depending on the types of probes (since conventional chips are used for a sole detection of DNAs or proteins); (3) high costs (since complicate immobilization procedure such as masking or printing for immobilizing the probes on the substrate is needed, and each chip can be used only once); and (4) low reliability (since when carrying on reactions with conventional chips, signals, which are not easily detected are generated on substrates, and background signals would interfere the detection).

In the development of biochips, a “biochemical reaction chip” that is different from the micro-array chip produced by micro-electro mechanical technology, is for a micro-amount of sample reacting in a chip such as polymerase chain reaction chip and capillary electrophoresis chip. For example, on the polymerase chain reaction chip, the surfaces of micro-wells are etched by micro-mechanical technology. A temperature control module is also set on a bottom or a back of the chip for adjusting the temperature in the wells and because of the small volume and large surface, the temperature in the wells changes easily. It shortens the reaction time of the polymerase chain reaction from several hours to several minutes. On the other hand, biochemical reaction chip has the following problems: (1) for different biochemical reactions, different chips are needed; which is not convenient; (2) it is difficult to perform further steps (such as purification) of the reaction solution because the volume of the reaction solution to be reacted on the biochemical reaction chip is quite small.

### SUMMARY OF THE INVENTION

The present invention provides a micro-array system for a micro amount of biomolecules carrying on a bioreaction. The micro-array system has the advantages of multi-purposes, short reaction time, easy post-manipulation, and easy process.

One objective of the present invention is to provide a micro-array system for a micro-amount of biomolecules carrying on a bioreaction in a reaction solution, which comprises a substrate comprising a plurality of micro-wells for receiving the reaction solution; a plurality of micro-beads placing in the reaction solution for the biomolecules attached on surfaces thereon; and a vibrating module for vibrating the substrate, which makes the biomolecules attached on the micro-beads react evenly. Optionally, the micro-array system further comprises a temperature control module for adjusting the temperature of the reaction solution.

The other objective of the invention is to provide a method for a micro-amount of biomolecules carrying on a bioreaction in a reaction solution, which comprises:

(a) providing a plurality of micro-beads;

(b) attaching the biomolecules onto the micro-beads;

(c) placing the micro-beads with the biomolecules attached thereon in the reaction solution; and

(d) placing the reaction solution into a plurality of micro-wells of a substrate, wherein the substrate is vibrated by a vibrating module to make the biomolecules attached on the micro-beads react evenly; optionally, the temperature of the reaction solution in the micro-wells is controlled by a temperature control module.

### BRIEF DESCRIPTION OF THE DRAWINGS

FIG. 1 shows a partial section view of the micro-array system according to the invention.

FIG. 2 shows a control circuit board of the micro-array system according to the invention.

FIG. 3 shows an electrophoresis result of TRAIL product synthesized by polymerase chain reaction using the micro-array system according to the invention; wherein 1 represents the reaction of free T7 and T3 primers (control group); 2 represents the reaction of free T7 primer and T3 primer conjugated on PolyScience® carboxylated magnetic beads; 3 represents the reaction of free T7 primer and T3 primer conjugated on BANG's Laboratory® carboxylated magnetic beads; 4 represents the reaction of free T3 primer and T3 primer conjugated on PolyScience® amine magnetic beads; 5 represents the reaction of free T3 primer and T7 primer conjugated on BANG's Laboratory®) amine magnetic beads; 6 represents the reaction of free T3 primer and T7 primer conjugated on Dynal® amine magnetic beads; and M represents molecular length marker.

FIG. 4 shows the fluorescence strength of DNA-DNA hybridization that takes Cy3 single stranded GADPH fragment as a target; wherein PN represents using PolyScience® amine magnetic beads; BN represents using BANG's Laboratory® amine magnetic beads; DN represents using Dynal® amine magnetic beads; a represents using the magnetic beads immobilized with single stranded DAPDH fragment as a probe; and b represents using the magnetic beads immobilized with single stranded TRAIL fragment as a probe.

FIG. 5 shows the quantification of HSA immobilized on different magnetic beads treated with different concentrations of EDC. In FIG. 5a, PL represents using PolyScience® amine magnetic beads; BN represents using BANG's Laboratory® amine magnetic beads; DN represents using Dynal® amine magnetic beads. In FIG. 5b, PL represents using PolyScience® carboxylated magnetic beads; and BN represents using BANG's Laboratory® carboxylated magnetic beads.

FIG. 6 shows the fluorescence strength corresponding to fluorescence labeled protein weight. FIG. 6a shows the fluorescence strength that is ten times the amount of the serious dilution of HSA or anti-HSA antibody. FIG. 6b shows the fluorescence strength corresponding to Cy 3 or Cy 0.5 labeled anti-HSA antibody; wherein Cy 3-Ab represents antibody labeled with Cy 3, and Cy 5-Ab represents antibody labeled with Cy 5. FIG. 6c shows the fluorescence strength corresponding to Cy 3 or Cy 5 labeled HSA antibody; wherein Cy 3-Ag represents antibody labeled with Cy 3, and Cy 5-Ag represents antibody labeled with Cy 5.

FIG. 7 shows the result of hybridization which takes 1 mg HSA immobilized on the magnetic beads as probes and fluorescence labeled anti-HSA antibody as targets. FIG. 7a shows the fluorescence strength of ten times serious dilution of hybridization. FIG. 7b shows the fluorescence strength corresponding to antibody amount, wherein the reaction solution is spotted on a glass for scanning. FIG. 7c shows the fluorescence strength of reaction solution adding with 1.6 μg of antibody. The signals are obtained by scanning the micro-wells; wherein Blank represents glass only, Control 1 represents HSA immobilized on the magnetic beads; Control 2 represents fluorescence labeled anti-HSA antibody; and +Ab represents the result of hybridization.

FIG. 8 shows the result of hybridization which takes 1 mg anti-HSA antibodies immobilized on the magnetic beads as probes and fluorescence labeled HSA as targets. FIG. 8a shows the fluorescence strength of ten times the amount of the serious dilution of hybridization. FIG. 8b shows the fluorescence strength of reaction solution adding with HSA. The signals are obtained by scanning the micro-wells; wherein Blank represents glass only, Control 1 represents anti-HSA antibodies immobilized on the magnetic beads; Control 2 represents fluorescence labeled HSA; and +Ab represents the result of hybridization.

FIG. 9 shows the result of hybridization which takes TATA primers immobilized on the magnetic beads as probes and fluorescence labeled nuclear proteins as targets. FIG. 9a shows the fluorescence strength of ten times serious dilution of hybridization. FIG. 9b shows the fluorescence strength of reaction solution in hybridization, wherein the reaction solution is spotted on a glass for scanning. FIG. 9c shows the fluorescence strength of reaction solution. The signals are obtained by scanning the micro-wells; wherein 1 represents probes immobilized on the magnetic beads, 2 represents the hybridization carried by the probes immobilized on the magnetic beads and nuclear proteins labeled with fluorescence; 3 represents the hybridization carried by the probes immobilized on the magnetic beads that are competed with 1× free probes and nuclear proteins labeled with fluorescence; and 4 represents the hybridization carried by the probes immobilized on the magnetic beads that are competed by 2× free probes and nuclear proteins labeled with fluorescence.

### DETAILED DESCRIPTION OF THE INVENTION

Combining micro-beads and biochemical reaction chip, the present invention develops a micro-array system that has the advantages of high sensitivity, easy post-manipulation, easy operation, high reliability and reusability.

According to the invention, the micro-array system is for a micro-amount of biomolecules carrying on a bioreaction in a reaction solution, which comprises a substrate comprising a plurality of micro-wells for receiving the reaction solution; a plurality of micro-beads placing in the reaction solution for the biomolecules attached on surfaces thereon; and a vibrating module for vibrating the substrate, which makes the biomolecules attached on the micro-beads react evenly. Optionally, the micro-array system further comprises a temperature control module for adjusting the temperature of the reaction solution.

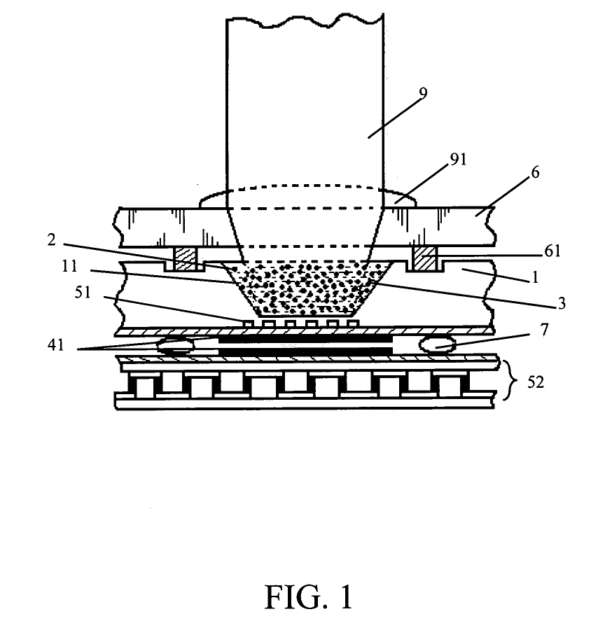

In one embodiment of the invention, the micro-array system (referring to FIG. 1) comprises (1) a substrate 1 comprising a plurality of micro-wells 11 for receiving a reaction solution 3; (2) a plurality of micro-beads 2 placing in the reaction solution 3 for biomolecules being attached on surfaces thereon; (3) a vibrating module (such as 41) for vibrating the substrate 1; and (4) a temperature control module (such as 51) for controlling the temperature of the reaction solution 3 in the micro-wells 11.

As used herein, the term “biomolecules” refers to molecules that involve in a bioreaction, which include but are not limited to macromolecules of nucleic acids, peptides and carbohydrates. The bioreaction suitable for being performed on the micro-array system according to the invention is the reaction that the biomolecules are involved in, which includes but is not limited to polymerase chain reaction, nucleic acid-nucleic acid hybridization, protein-protein hybridization, and nucleic acid-protein hybridization. Preferably, the reaction further comprises the step of controlling a precise temperature, or isolating the biomolecules from the reaction solution.

In one embodiment of the invention, the micro-array system may be applied in hybridization, such as nucleic acid-nucleic acid hybridization, protein-protein hybridization, and nucleic acid-protein hybridization. Such hybridization takes a biomolecule (such as nucleic acid or protein) as a probe to combine a target for obtaining a detectable signal. The combination of the probe and target contains base pairing, protein-protein interaction, nucleic acid-protein interaction, or interaction between biomolecules. The combination usually occurs under an appropriate condition such as an appropriate temperature, an appropriate ion strength or an appropriate pH value. The combination is terminated by changing the condition physically (such as changing the temperature) or chemically (such as changing pH value or ion strength). Then, the probe biomolecule is separated from the target. Therefore, the probe can be reused. That is, the micro-array system according to the invention has an advantage of being reusable.

As used herein, the term “a substrate” refers to a support containing a plurality of micro-wells and can be set up with a temperature control module and a vibrating module. The material of the substrate is biologically inert with respect to the biomolecules according to the invention. For example, it is made from silicon. The micro-wells on the substrate may be formed by any conventional technology. In one embodiment of the invention, the substrate is made from silicon and the micro-wells are defined by photolithography and then etched by conventional technologies, such as the method using potassium hydroxide, or an inductive coupling plasma dry etching technology. The volume and amount of the micro-wells are designed on the basis of the reaction volume and the reaction number in a test.

As used herein, the term “micro-bead” refers to a small bead of which a surface can be attached to biomolecules or can be attached after an appropriate activation. Preferably, the micro-beads can be isolated from the reaction solution easily for the purpose of isolating the biomolecules immobilized thereon from the reaction solution. In one embodiment of the invention, the micro-beads are magnetic beads which can be separated by magnetic force. The method of immobilizing of the biomolecules on the micro-beads is any suitable method known by persons skilled in this art. For example, the surfaces of the micro-bead are treated to have a carboxyl group for forming an amide bond with the biomolecule having an amino group, or the micro-beads are treated to have an amino group for forming an amide bond with the biomolecule having a carboxyl group. In one embodiment of the invention, the surfaces of the micro-beads are activated with a coupling agent such as 1-ethyl-3-(3-dimethylaminopropyl)carbodiimide hydrochloride (EDC) for the biomolecules immobilized thereon.

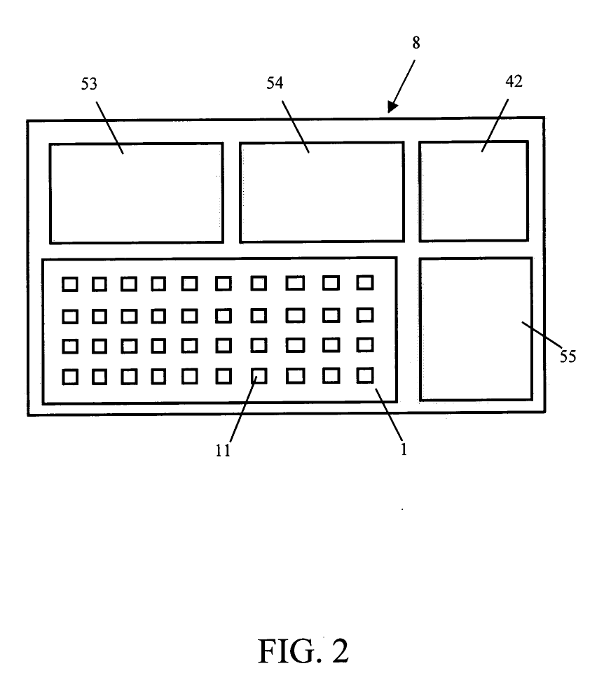

As used herein, the term “vibrating module” refers to an apparatus for vibrating the substrate to make the micro-beads in the micro-wells react evenly in the reaction solution. Referring to FIGS. 1 and 2, in one embodiment of the invention, the vibrating module comprises an electrostatic vibrator 41 set under the substrate 1 and a vibration control circuit 42. The electro-static vibrator 41 may be fabricated from two metal parallel plates using MEMS technology. The vibration control circuit 42 may be fabricated using CMOS technology.

As used herein, the term “temperature control module” refers to an apparatus for controlling the temperature of the reaction solution in the microwells. In one embodiment of the invention (referring to FIGS. 1 and 2), the temperature control module comprises a temperature sensor for detecting the temperature of the reaction solution 3, the temperature sensor being controlled by a sensing circuit 53; a heater for heating the reaction solution 3, the heater being controlled by a heating circuit 54; and a cooler for cooling the reaction solution 3 (such as a thermal-electrical cooler, TE cooler), the cooler being controlled by a cooling control circuit 55. In one embodiment of the invention, the temperature sensor and the heater may be integrated in a heating/sensing resistor 51. The temperature sensor 51, heater 51 and cooler 41 are fabricated by artisans skilled in this art using MEMS technology. For example, the heating/sensing resistor 51 is fabricated by boron diffusion under the micro-wells 11. The sensing circuit 53, heating circuit 54 and cooling control circuit 55 are fabricated by CMOS technology. These circuits 53, 54 and 55 may be connected by a metal bump 7 and further connected with the heating/sensing resistor 51 and TE cooler 52. Besides, the metal bump 7 provides a thermal conductor sandwiched between the TE cooler 52 and the substrate 1. The temperature control module for controlling the temperature of bioreaction comprises a temperature sensor for sensing the temperature of the reaction solution 3, and the heater 51 or cooler 52 for adjusting to the desired temperature. In view of the small volume of the reaction solution 3, the reaction solution can be quickly heated or cooled so as to speed the reaction and enhance the stability by a temperature control module. The circuits 53, 54 and 55 and the vibration control circuit 42 may be integrated in a control circuit board 8.

Optionally, when the biomolecules need laser stimulation, the micro-array system according to the invention further comprises a laser source 9, preferably, a micro-lens 91. The laser source 9 is micro-adjusted by the micro-lens 91 to focus precisely on the area of the micro-wells 11.

In one embodiment of the invention, the micro-array system further comprises a signal detector for detecting signals obtained in the micro-wells 11. For example, the signal detector comprises a scanner for transferring the signal to a signal processor for further analysis.

In one embodiment of the invention, the micro-array system further comprises a cover plate 6 fixed on the substrate 1 by a seal 61. The cover plate 6 prevents instability of the agents in the reaction solution due to the evaporation of the reaction solutions 3 during the reaction, especially the heating reaction. For example, the cover plate 6 is a 7740 glass, and the seal 61 is a polyimide seal.

The invention further provides a method for a micro-amount of biomolecules carrying on a bioreaction in a reaction solution, which comprises:

(a) providing a plurality of micro-beads;

(b) attaching the biomolecules onto the micro-beads;

(c) placing the micro-beads with the biomolecules attached thereon in the reaction solution; and

(d) placing the reaction solution into a plurality of micro-wells of a substrate, wherein the substrate is vibrated by a vibrating module to make the biomolecules attached on the micro-beads react evenly whereby; optionally, the temperature of the reaction solution in the micro-wells is controlled by a temperature control module.

The micro-array system and the method according to the invention have the advantages of (1) being easily opearated, (2) having a high sensitivity, (3) being applied for multi-purposes, (4) having a high reliability, (5) easily being post-manipulated, and (6) being reusable. According to the invention, the process for immobilizing the biomolecules on the micro-beads is much easier than that for immobilizing the probes on the substrate in the conventional micro-chips. In the method of the invention, only one single step is needed, and the costs are lowered. The biomolecules immobilized on the micro-beads react evenly in the reaction solution by the vibrating module. Therefore, the micro-array system according to the invention has a higher sensitivity in comparison with the conventional chips that immobilize probes on substrate plates. In addition, the micro-array system according to the invention is suitable for different biomolecules such as nucleic acids, peptides or carbohydrates. On the contrary, the chips suitable for different targets to be tested are necessary for different biomolecules in the prior art. In the bioreaction carried out in the micro-wells according to the invention, the signal area (i.e., the micro-well) and the background area are clearly defined during the signal detection. Positioning can be improved by detecting the micro-wells, and thus the reliability is enhanced. In view of the biomolecules immobilized on the micro-beads, the biomolecules are easily isolated from the reaction solution due to the easy operation of micro-beads. When carrying on hybridization, the biomolecules immobilized on the micro-beads can be separated from the targets physically or chemically after completing the reaction, and thus the system can be reused.

The following examples are given for the purpose of illustration only and are not intended to limit the scope of the present invention.

### Example 1

### Micro-Array System

Referring to FIGS. 1 and 2, the micro-array system in the example comprises a substrate 1 comprising a plurality of micro-wells 11; a plurality of micro-beads 2, an electro-static vibrator 41, a heating/sensing resistor 51 and a TE cooler 52 controlled by a vibration control circuit 42, a sensing circuit 53, a heating circuit 54, and a cooling control circuit 55 integrated in a control circuit board 8; a plurality of metal bump 7, and a 7740 glass cover plate 6 sealed by a polyimide seal 61.

### Example 2

### Polymerase Chain Reaction

Primer Immobilization: The amine or carboxylated beads were washed with 0.2 M of 2-(N-morpholino)ethanesulfonic acid (MES), pH 5.0 buffer. The magnetic beads were purchased from PolyScience®, BANG's Laboratory® and Dynal®.

T3 or T4 primers were modified to carry carboxyl group or amino group at their 5′ ends.

10 mg of washed magnetic beads, 200 μg of modified primers, and 0.05 M of 1-ethyl-3-(3-dimethylaminopropyl)-carbodiimide hydrochloride (EDC) were shaken for 60 minutes at room temperature. EDC was removed by attracting the magnetic beads by a magnet. The beads were washed twice with a wash buffer (20 mM NaH₂PO₄/Na₂HPO₄ pH7.5, 0.5% Tween 20) and stored in a storage buffer (20 mM NaH₂PO₄ pH7.5, 0.1% w/v bovine serum albumin, 0.02% sodium azide) in the concentration of 20 mg/mL.

Polymerase Chain Reaction: T3 or T7 primers immobilized on the magnetic beads were washed twice with water. Polymerase chain reactions were conducted by T3 and T7 primers in a free form or immobilized on the magnetic beads, the plasmids containing TNF-related apoptotosis inducing ligand (TRAIL) or glyceraldehydes phosphate dehydrogenase (GAPDH) ESTs as templates. Fifty-μL reaction solution contained 0.5 μL template, each 500 nM of T3 primer immobilized on the magnetic beads and free T7 primer or T7 primer immobilized on the magnetic beads and free T3 primer, each 250 μM dNTP, 1 U Taq polymerase (purchased from Finnzymes®, Fin, Espoo), 10 mM Tris-HCl, 1.5 mM magnesium chloride, 150 mM potassium chloride, and 0.1% Triton X-100.

The reaction solutions containing magnetic beads were transferred to the micro-wells in Example 1, and the temperature was controlled at 95° C. for 3 minutes, 35 cycles of 95° C. for 30 seconds, 55° C. for 40 seconds and 72° C. for 1 minute, and followed with 72° C. for 5 minutes by the temperature control module. The reaction solutions were vibrated by an electro-static vibrator during the reaction.

A control group was prepared by using free T3 and T7 primers under the same conditions with a conventional polymerase chain reaction machine.

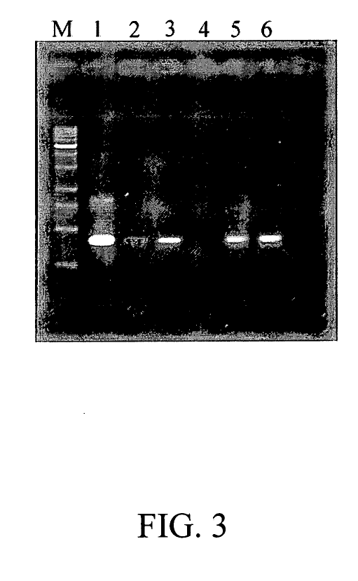

Result: The reaction product of the TRAIL EST plasmid was subject to 1% agarose gel electrophoresis and the results were shown in FIG. 3. Except the reaction using T7 primer immobilized on amine PolyScience® magnetic beads and free T3 primer (FIG. 3, Lane 4), the desired TRAIL fragments were obtained in every reaction.

### Example 3

### DNA-DNA Hybridization

Probe Preparation: The magnetic beads immobilized with the DNA fragments containing TRAIL or GAPDH were prepared as described in Example 2. The magnetic beads used were PolyScience® amine magnetic beads, BANG's Laboratory® amine magnetic beads and Dynal® amine magnetic beads. The magnetic beads containing the DNA were heated to 95° C. to obtain the magnetic beads containing single stranded DNA as a probe in the hybridization.

Target Preparation: Except for Cy 3-dUTP (purchased from AP Biotech®, Uppsala, Sweden) replacing 50% of dTTP for obtaining fluorescence DNA fragment, the targets were synthesized using free T3 and T7 primers and GAPDH EST plasmid as template according to the condition described in Example 2 with a conventional polymerase chain reaction machine. The fragments were heated to 95° C. to obtain single stranded DNA as a target.

Hybridization: The hybridization solution containing 6× saline-sodium citrate (SSC), 0.5% SDS and 5× Denhard's solution (0.1% BSA, 0.1% Ficoll, 0.1% polyvinykpyrrolidone) was added with 50 ng single stranded GADPH fragment and 2 μL of 20 mg/mL magnetic beads immobilized with TRAIL or GAPDH single stranded fragments. The reaction solutions were transferred into the micro-wells of the micro-array system in Example 1 for reacting at 65° C. for 5 hours and vibrated by the electro-static vibrator. The magnetic beads were isolated with a magnet and washed twice at room temperature with the wash buffer (0.1% SSC and 0.5% SDS).

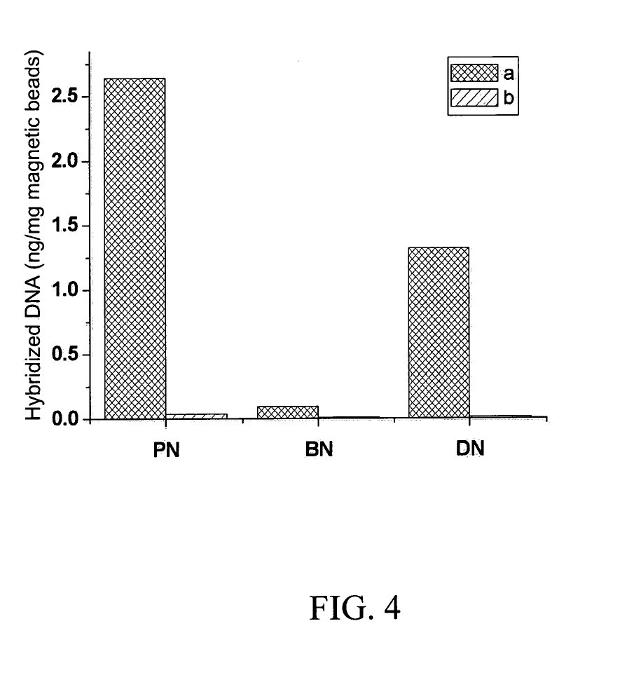

Signal Detection and Result: The reaction solutions were spotted on a glass and scanned with Axon® scanner and the fluorescence images were analyzed with ScanAlyze® software (http://www.microarrays.org/software). The result is shown in FIG. 4.

In the hybridization taking single stranded GADPH as the probe (FIG. 4, group a), PolyScience® amine magnetic beads and Dynal® amine magnetic beads groups were detected to have fluorescence signals, but the signal was weak in the BANG's Laboratory® amine magnetic group. Besides, in the control group taking single stranded TRAIL as the probe (FIG. 4, group b), the signals were all quite weak in the groups of these three beads. It showed high specificity and sensitivity of the DNA-DNA hybridization using the micro-array system according to the invention.

### Example 4

### Protein-Protein Hybridization

Protein Probe Preparation: Ten μL of amine or carboxylated beads were washed with 0.2 M of MES, pH 5.0 buffer. The magnetic beads were purchased from PolyScience®, BANG's Laboratory® and Dynal®. The washed magnetic beads, 20 μg of human serum albumin (HSA) or anti-HSA, and 0, 50, 100, or 200 mM of EDC were shaken for 30 minutes at room temperature. EDC was removed by attracting the magnetic beads by a magnet. The beads were washed twice with 1 M sodium chloride and then once with 10 mM Tris-HCl (pH 8.0) and stored in 10 μL Tris-HCl (pH 8.0).

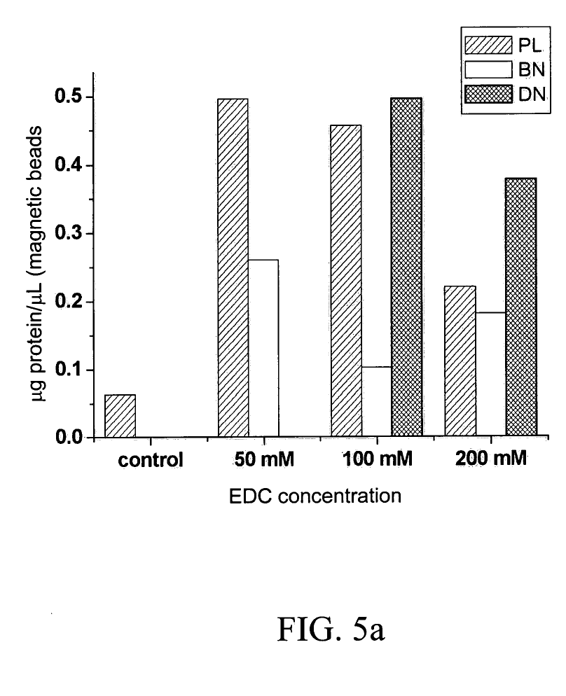

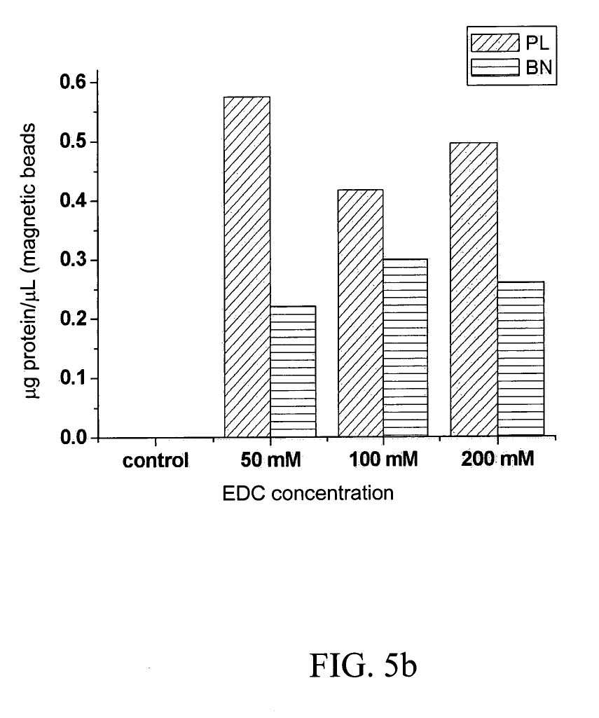

The amounts of HSA protein immobilized on different magnetic beads were measured with BCA assay (Sigma®) in the treatment of different concentrations of EDC. FIG. 5a shows the quantification of HSA immobilized on PolyScience®, BANG's Laboratory® and Dynal® amine magnetic beads. FIG. 5b shows the quantification of HSA immobilized on PolyScience® and BANG's Laboratory® carboxylated magnetic beads.

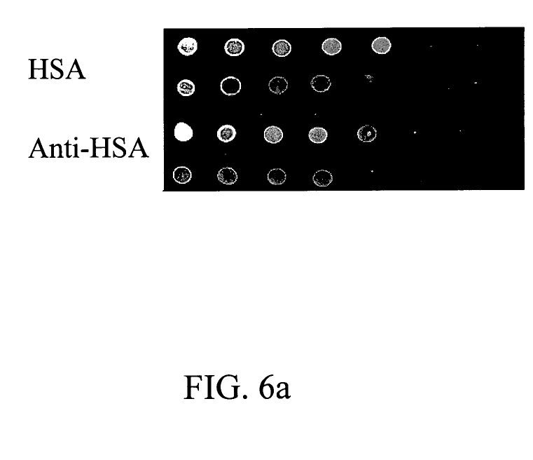

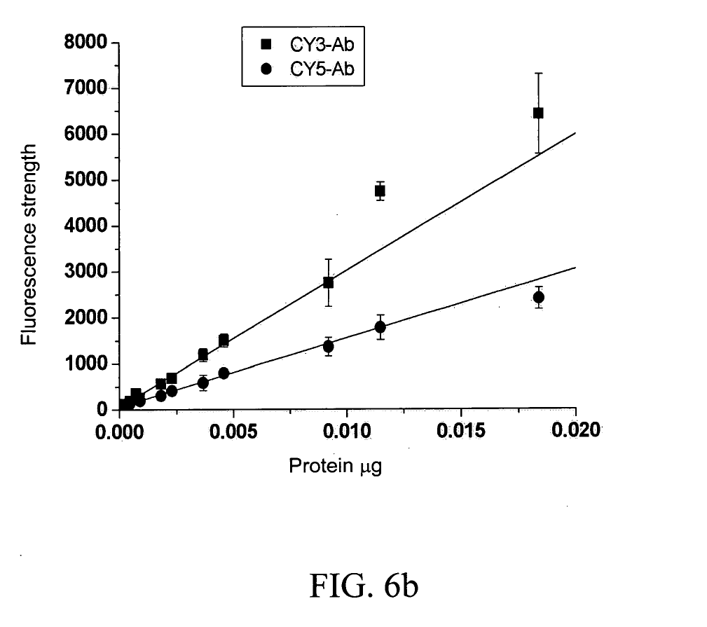

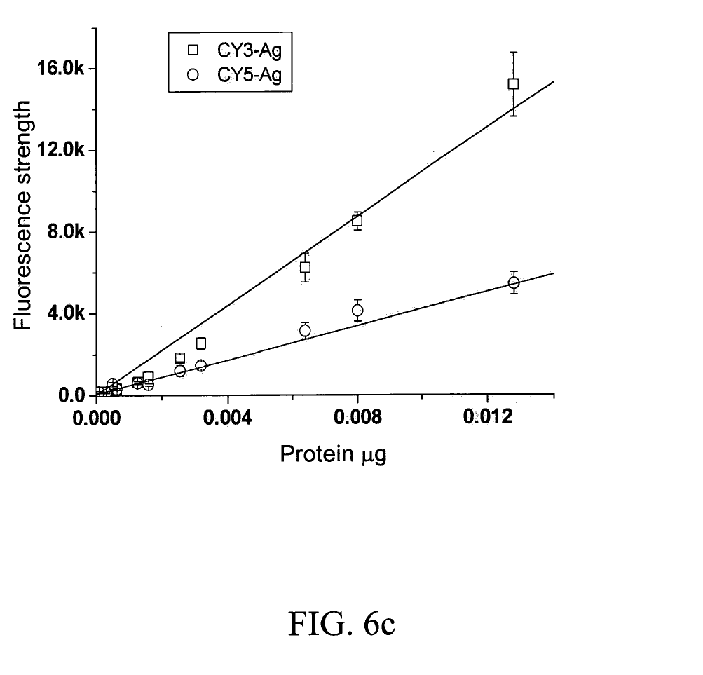

Target Proteins Preparation. HSA or anti-HSA proteins were in 0.1 M sodium carbonate pH 8.0 buffer, and mixed with NHS-ester activated Cy 3 or Cy 5 fluorescence dyes (purchased from Amersham® catalog # PA23001 and PA25001) in the final protein concentration of 2 mg/mL and dye concentration of 300 μM. The reaction was conducted at room temperature in dark for 45 minutes and terminated by adding 1 M Tris-HCl pH 8.0. The free dyes were removed by centrifugation and the protein concentration was adjusted to 2 mg/mL. The fluorescence strength corresponding to protein weight is shown in FIG. 6. FIG. 6a shows the fluorescence strength of ten times serious dilution of HSA or anti-HSA antibody spotted on poly-L-lysine coated glass, which was scanned with an scanner and analyzed with GenePix™. FIGS. 6b and 6c show the fluorescence strength corresponding to Cy 3 or Cy 5 labeled anti-HSA antibody or HSA. It demonstrated that the fluorescence strength and protein weight were in about-linear relation.

Hybridization: The protein probe immobilized on the magnetic beads was placed in the micro-wells of the micro-array system in Example 1, and added with 3% non-fat TTBS solution (20 mM Tris-HCl pH 7.4, 0.5 M NaCl, 0.5% Tween 20) for blocking for 1 hour, and then washed twice with TTBS solution. Different concentrated fluorescence labeled target proteins were then added and reacted at 37° C. for 1 hour. The supernatant was then removed and washed three times with TTBS solution without milk. The fluorescence was detected by putting the magnetic beads in 0.1 M Tris buffer. The reactions mentioned above were vibrated by the electro-static vibrator.

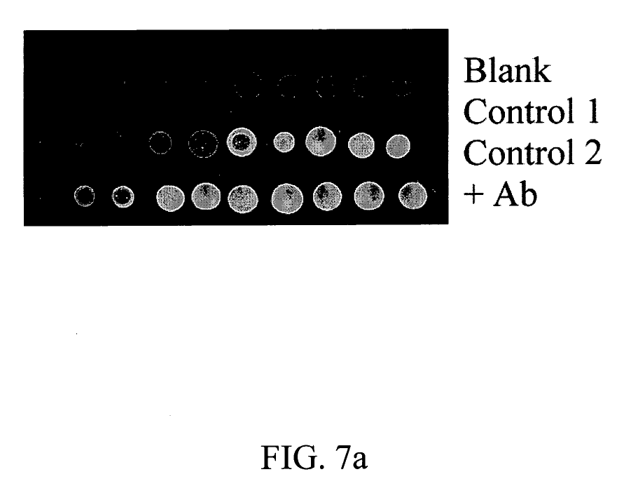

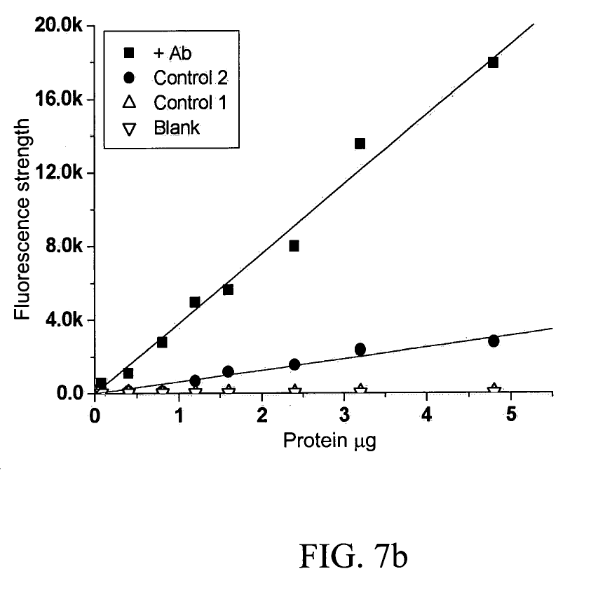

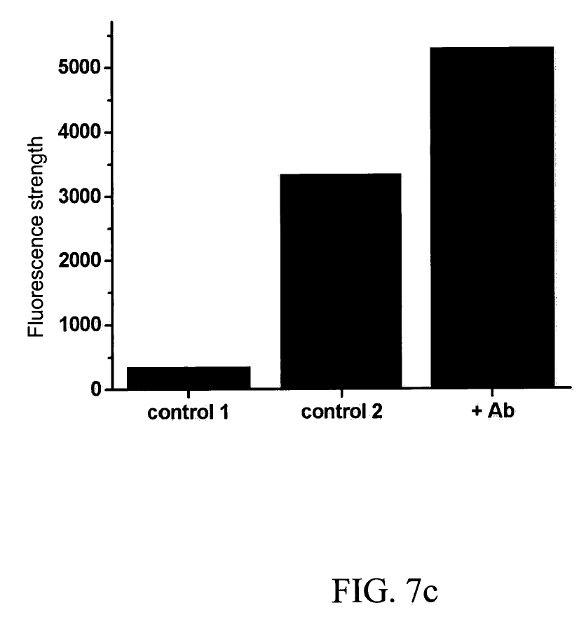

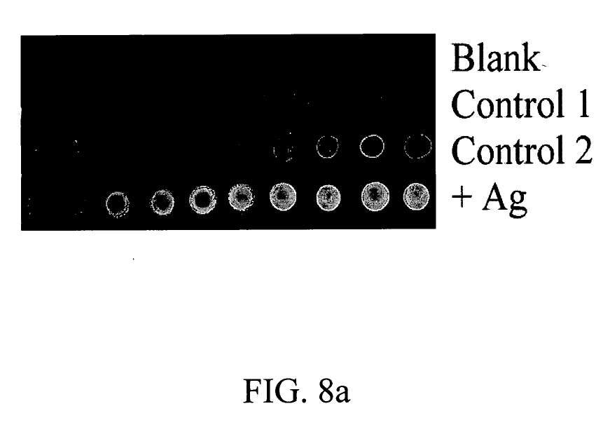

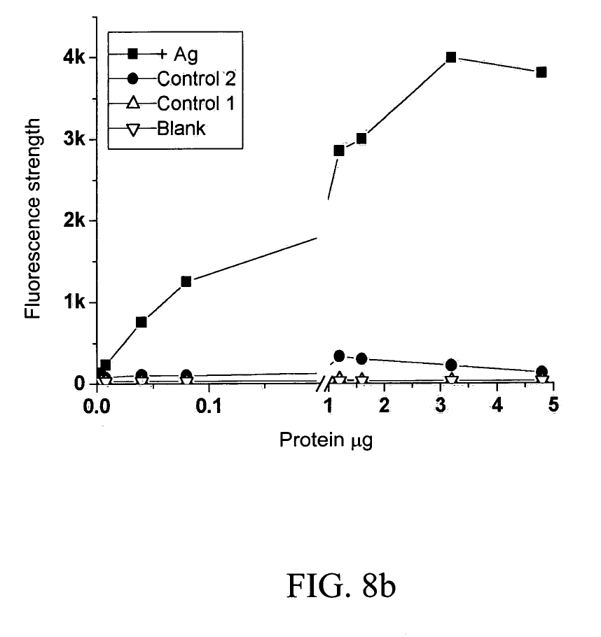

Signal detection and result: The result of hybridization which took 1 mg HSA immobilized on the magnetic beads as a probe and the fluorescence labeled anti-HSA antibody as a target is shown in FIG. 7. FIG. 7b shows the reaction solution spotted on a glass and scanned by Axon® scanner. FIG. 7c shows the fluorescence signal in the micro-wells detected by a scanner and analyzed with ScanAlyze® software. The result of hybridization taking 1 mg anti-HSA antibody immobilized on the magnetic beads as a probe and fluorescence labeled HSA as a target is shown in FIG. 8.

Referring to FIG. 7, the fluorescence signal increased with the increasing amount of antibody in about linear relation. It showed high specificity and sensitivity of the micro-array system. However, in FIG. 8, no significant correlation is observed between antibody probe and target antigen. The reason might be that the effect of immobilizing the large antibody was not good enough.

### Example 5

### Double Stranded DNA-Protein Hybridization

Double-Stranded DNA Probe Preparation. The preparation of double-stranded DNA probe was similar to the method of probe immobilization in Example 2; wherein the primer immobilized was TATA primer.

Target Protein Preparation: The target protein was nuclear protein extracted from human non-small lung cancer cell H460 and prepared similarly to the method of target protein preparation in Example 4.

Hybridization: The hybridization was performed in 20 μL of binding buffer (5 mM HEPES, pH 7.9, 100 mM KCl, 3 mM MgCl₂, 1 mM EDTA, 0.5 mM DTT, 10% glycerol and 2 mg poly (dI-dC)). The TATA primer immobilized on magnetic beads was blocked in 3% non-fat milk at 37° C. for 1 hour, and then washed twice with TTBS solution. Different concentrated fluorescence labeled target proteins were then added and reacted at 37° C. for 20 minutes. The supernatant was removed and washed three times with TTBS solution without milk. The fluorescence was detected by putting the magnetic beads in 0.1 M Tris buffer. The experiments were performed by pre-interacting the target proteins with free primer probe for 10 minutes and then reacting with the probe immobilized on the magnetic beads. The reactions mentioned above were vibrated by the electro-static vibrator.

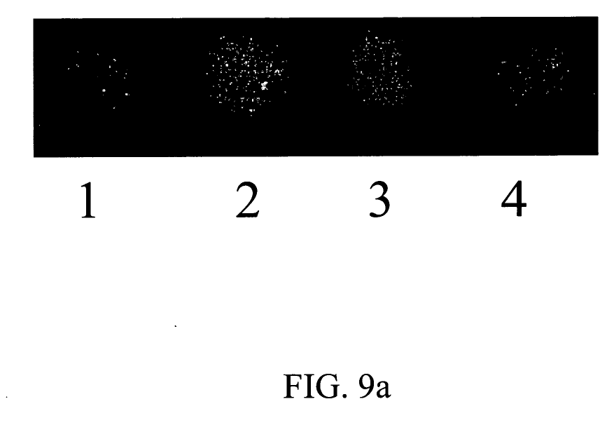

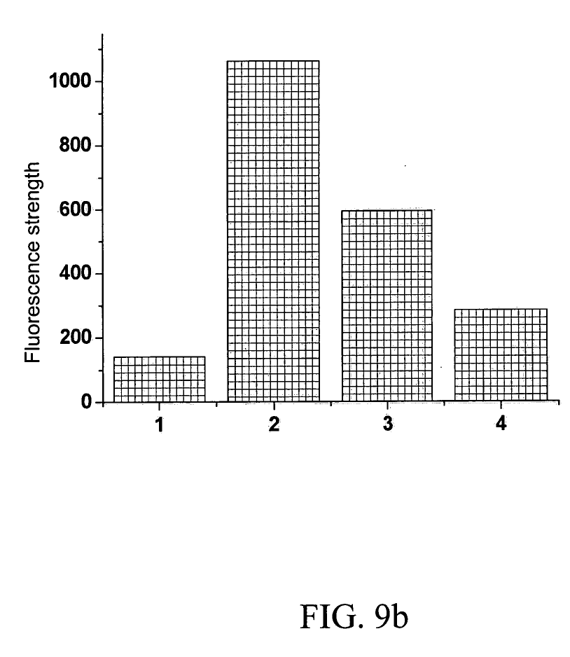

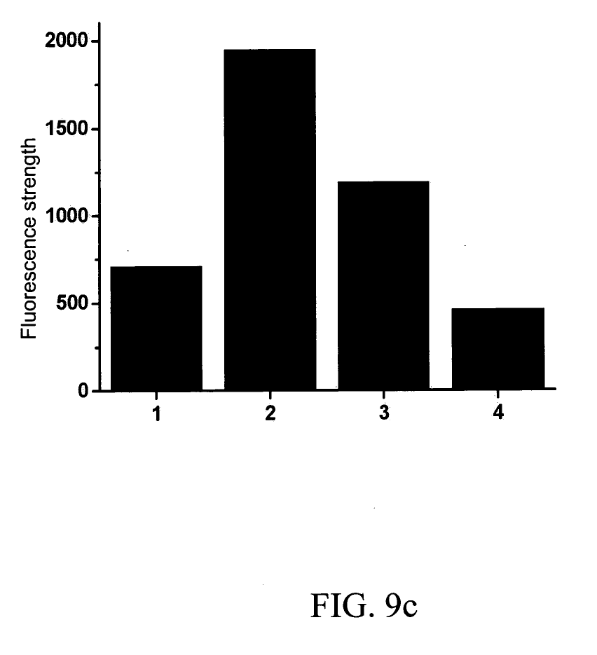

Signal Detection and Result: The results of signal detection and analysis are shown in FIG. 9. FIG. 9b shows the reaction solution spotted on a glass and scanned by Axon® scanner. FIG. 9c shows the fluorescence signal in the micro-wells detected by a scanner and analyzed with ScanAlyze® software. The fluorescence signal decreased with the increasing competing probes. It showed that the -array system might be applied in DNA-protein hybridization.

While embodiments of the present invention have been illustrated and described, various modifications and improvements can be made by those skilled in the art. The embodiments of the present invention are therefore described in an illustrative but not restrictive sense. It is intended that the present invention is not limited to the particular forms as illustrated, and that all the modifications not departing from the spirit and scope of the present invention are within the scope as defined in the appended claims.

What is claimed is:

1. A micro-array system for a micro amount of biomolecules carrying on a bioreaction in a reaction solution, which comprises:
a substrate comprising a plurality of micro-wells for receiving the reaction solution;
a plurality of micro-beads placing in the reaction solution for the biomolecules attached on surfaces thereof; and
a vibrating module for vibrating the substrate, which makes the biomolecules attached on the micro-beads react evenly.

2. The micro-array system according to claim 1, wherein the biomolecules are selected from the group consisting of nucleic acids, peptides and carbohydrates.

3. The micro-array system according to claim 1, wherein the bioreaction is selected from the group consisting of polymerase chain reaction, nucleic acid-nucleic acid hybridization, protein-protein hybridization, and nucleic acid-protein hybridization.

4. The micro-array system according to claim 1, wherein the substrate is made from silicon.

5. The micro-array system according to claim 1, wherein the micro-beads are magnetic beads.

6. The micro-array system according to claim 1, wherein the micro-beads are activated with a coupling agent for the biomolecules immobilized thereon.

7. The micro-array system according to claim 6, wherein the coupling agent is 1-ethyl-3-(3-dimethylaminopropyl)-carbodiimide hydrochloride.

8. The micro-array system according to claim 1, wherein the vibrating module is set under the substrate.

9. The micro-array system according to claim 1, wherein the vibrating module comprises an electro-static vibrator.

10. The micro-array system according to claim 1 further comprising a temperature control module for controlling the temperature of the reaction solution.

11. The micro-array system according to claim 10, wherein the temperature control module comprises a temperature sensor, a heater, and a cooler.

12. The micro-array system according to claim 11, wherein the temperature sensor and the heater are a heating/sensing resistor.

13. The micro-array system according to claim 1 further comprising a laser source.

14. The micro-array system according to claim 13 further comprising a lens.

15. The micro-array system according to claim 1 further comprising a cover plate.

16. The micro-array system according to claim 1 further comprising a signal sensor.

17. A method for a micro amount of biomolecules carrying on a bioreaction in a reaction solution, which comprises:
(a) providing a plurality of micro-beads;
(b) attaching the biomolecules onto the micro-beads;
(c) placing the micro-beads with the biomolecules attached thereon in the reaction solution; and
(d) placing the reaction solution into a plurality of micro-wells of a substrate, wherein the substrate is vibrated by a vibrating module to make the biomolecules attached on the micro-beads react evenly.

18. The method according to claim 17, wherein the biomolecules are selected from the group consisting of nucleic acids, peptides and carbohydrates.

19. The method according to claim 17, wherein the bioreaction is selected from the group consisting of polymerase chain reaction, nucleic acid-nucleic acid hybridization, protein-protein hybridization, and nucleic acid-protein hybridization.

20. The method according to claim 17, wherein the substrate is made from silicon.

21. The method according to claim 17, wherein the micro-beads in step (a) are magnetic beads.

22. The method according to claim 17, wherein the micro-beads in step (b) are activated with a coupling agent for the biomolecules immobilized thereon.

23. The method according to claim 22, wherein the coupling agent is 1-ethyl-3-(3-dimethylaminopropyl)-carbodiimide hydrochloride.

24. The method according to claim 17, wherein the vibrating module is set under the substrate.

25. The method according to claim 17, wherein the vibrating module comprises an electro-static vibrator.

26. The method according to claim 17, wherein the temperature of the substrate is controlled by a temperature control module for controlling the temperature of the reaction solution in the micro-wells.

27. The method according to claim 26, wherein the temperature control module comprises a temperature sensor, a heater, and a cooler.

28. The method according to claim 17, wherein the temperature sensor and the heater are a heating/sensing resistor.

29. The method according to claim 17 further comprising activating the reaction solution with a laser source.

30. The method according to claim 29 further comprising adjusting the laser source with a lens.

31. The method according to claim 17 further comprising applying a cover plate during the bioreaction.

32. The method according to claim 17 further comprising monitoring the bioreaction with a signal sensor.
